(19) United States
(12) Patent Application Publication
(10) Pub. No.: US 2023/0404827 A1
(43) Pub. Date: Dec. 21, 2023

(54) Apparatus and Method for Capturing Aerosols

(71) Applicant: The Board of Regents of the University of Oklahoma, Norman, OK (US)

(72) Inventors: Fernando Luis Esteban Florez, Oklahoma City, OK (US); Tyler G. Thibodeau, Norman, OK (US); Paul M. Mullasseril, Edmond, OK (US); Sharuhk S. Khajotia, Edmond, OK (US); C. Rieger Wood, III, Tulsa, OK (US); Michael Ferguson, Norman, OK (US)

(73) Assignee: The Board of Regents of the University of Oklahoma, Norman, OK (US)

(21) Appl. No.: 18/037,706

(22) PCT Filed: Nov. 7, 2021
(86) PCT No.: PCT/US2021/058370

§ 371 Date: May 18, 2023

Related U.S. Application Data
(60) Provisional application No. 63/110,982, filed on Nov. 7, 2020.

(51) Int. Cl. A61G 10/00; A61G 15/10

(52) U.S. Cl. CPC A61G 10/005; A61G 15/10

(57) ABSTRACT

An aerosol containment device is designed for use in containing aerosols discharged during a dental procedure. In some embodiments, the aerosol containment device includes a vacuum source, a vacuum hose connected to the vacuum source, a manifold connected to the vacuum hose, and a clear plastic shield connected to the manifold. The manifold includes a front inlet, a rear discharge connected to the vacuum hose, and a plenum extending between the front inlet and the rear discharge. In some embodiments, the manifold includes a plurality of entry vanes adjacent to the front inlet and a plurality of interior foils connected to the entry vanes. In other embodiments, the manifold includes a plurality of entry vanes adjacent to the front inlet and a plurality of interior vanes connected to the entry vanes. In other embodiments, the manifold does not include entry vanes, interior vanes, or interior foils.

### RELATED APPLICATIONS

The present application claims the benefit of U.S. Provisional Patent Application Ser. No. 63/110,982 filed Nov. 7, 2020 entitled, “Apparatus and Method for Capturing Aerosols,” the disclosure of which is incorporated by reference as if fully set forth herein.

### BACKGROUND

In December of 2019, Chinese health authorities reported on a new type of zoonotic infectious respiratory disease. The pathogen was soon thereafter identified as SARS-CoV-2 (severe acute respiratory syndrome coronavirus 2) and the resulting infection was named corona virus disease 2019 (COVID-19). Coronaviruses such as SARS-CoV (severe acute respiratory syndrome), MERS-CoV (Middle East Respiratory Syndrome) and SARS-CoV-2 cause a broad range of diseases in birds and mammals. In humans, coronaviruses are known to cause potentially lethal (mortality rates: 2% to 15%) pneumonia-like infectious diseases that are clinically translated into patients displaying flu-like symptoms including headache, fever, cough, myalgia, fatigue, abnormal chest CT scans, sputum production, hemoptysis and diarrhea. The common routes for COVID-19 spreading include (i) airborne transmission through inhalation of droplets and aerosols and (ii) direct contact transmission from exposure of conjunctival, nasal or oral mucosa to contaminated body fluids.

Droplet nuclei (1-5 μm), aerosols (<50 μm), droplets and splatter (>50 μm) are terms used interchangeably to commonly describe gas-based colloidal suspensions containing dispersed particles (either liquid [water, saliva, blood, sputum] or solid [bacteria, virus, fungus and dental plaque]). These suspensions can be generated by humans, animals and different types of instruments (e.g., rotary, oscillating, vibrating, piezosurgical, nebulizing, etc.). According to previous studies, particles sizes are typically associated with the generation process (e.g., combustion, mechanical or biological), and particle sizes produced, not only impact particle's ability to become suspended in air, but also influences its behavior in air such as settling, impaction and coagulation. In health-care facilities, nosocomial spreading of pathogens may occur when patients generate respirable particles (diameters <4.0 μm) by sneezing and coughing, or when pathogens become reaerosolized after being deposited on surfaces.

In dentistry, the utilization of rotary instruments (low-speed or high-speed handpieces), ultrasonic scalers and air-water syringes results in the formation of bioaerosols containing a broad variety of microorganisms and viruses. In fact, previous scientific evidence has indicated that the potential for contamination through an aerosol depends on the amount and quality of saliva, nasal and throat secretions, and the presence of blood, dental plaque or oral infections (e.g., caries, periodontitis, abscesses, etc.). Other studies have demonstrated that SARS-CoV-2 viral load in the oronasal pharynx tend to vary between 10²-10¹¹ copies/mL of respiratory fluid where the highest concentrations detected were typically found at the onset of COVID-19 symptoms.

In a study by Kobza et al. (Kobza, J., Pastuszka, J. & Br goszewska, E. Do exposures to aerosols pose a risk to dental professionals? Occup Med (Lond) 68, 454-458 (2018)), bioaerosols exposed to dental professionals were shown to have bacterial exposures that varied from 1.86×10⁵ bacteria/m³ to 4.3×10⁵ bacteria/m³ depending on the type of procedure conducted (cavity preparation [24-105 CFU/m³]>ultrasonic scaling [42-71 CFU/m³]>oral examination [24-62 CFU/m³]). Results reported indicate that bacteria (17 species, 10 sub-types; most prevalent: Staphylococci and Bacilli) and mold fungi (7 species, 4 sub-types; most prevalent: Penicillium and Cladosporium) were the predominant types of microorganisms found in dental bioaerosols. Another study demonstrated that ultrasonic scalers were capable of generating splatter even without the utilization of water coolant, and airborne material, spread over a distance of at least 18 inches from the operatory site.

According to the U.S. National Institute for Occupational Safety and Health (NIOSH), particles with dimensions similar to those found to contain peak levels of SARS-CoV-2 (between 0.25 μm and 0.5 μm) may remain suspended in air (either still or turbulent) for as long as 41 hours, which favors their ability to transmit infectious respiratory diseases like COVID-19, tuberculosis, influenza and others. In vitro studies investigating the stability and persistency of SARS-CoV-2 in aerosols and surfaces (plastic, stainless steel, copper and cardboard) have indicated, through the utilization of a Bayesian regression model, that SARS-CoV-2 can remain infectious while airborne for extended periods of times. In this critical scenario, several attempts to reduce the exposure of dental professionals to bioaerosols with pathogenic potential have been previously reported. These strategies include preprocedural microbial control using antimicrobial photodynamic therapy, mouthwash rinse solutions (0.12% chlorhexidine gluconate, cetylpyridinium chloride), high-volume evacuators, in-service instrumentation coolant agents and antiseptic agents dispensed directly into the dental unit waterlines (DUWLs). Since 1996, the Council on Scientific Affairs and the Council on Dental Practice of the American Dental Association have included recommendations for the control of bioaerosols that encompass the utilization of personal protective equipment (PPE), rubber dams and appropriate positioning of patients. Preprocedural mouth rinse with tempered chlorhexidine (47° C.) has been shown to be the most effective strategy to reduce aerosol-related bacterial load in dental operatories. Based on the context presented, it becomes obvious that characterization of the physical and chemical properties of aerosols in complex atmospheres is of fundamental importance to determine aerosol impact in human health and for the development of effective engineering controls.

Several methods are available for measuring particulate mass concentrations including filtration, beta gauges, gravimetric mass, tapered element oscillating microbalance, condensation particle counter, differential and scanning mobility analyzers, optical counters, aerodynamic particle sizers and aerosol mass spectrometers. Among the quantification approaches cited, aerodynamic particles sizing is preferred over light scattering methods because it measures particle size based on behavior in air, has a fast response time and detailed resolution of particles in the most important size range (0.3 μm to 20 μm). Despite recent developments in the field of aerosol measurement and control, the capability to accurately determine the spatial spreading of aerosols with respect to particle size under defined experimental conditions is still limited.

In dentistry, handheld instruments (turbines, ultrasonic scalers and air/water syringes) are considered the primary source of aerosols and splatter. A recent study investigating the topographical aspects of airborne contamination (on the dental chair and operatory room) caused by dental handpieces in clinical settings has demonstrated that contamination levels significantly varied based on the dental chair (p<0.01) and operatory room (p<0.0001) in function of the type of handpiece used (dental chair=air turbine [0.51±0.17]>scaler [0.47±0.14]>contra-angle [0.41±0.14]; operatory room=air turbine [0.26±0.38]>contra-angle [0.20±0.26]>0.17±0.27). Findings also indicated that contamination levels were high and fairly well distributed on the ceiling and walls of the operatory room, thereby suggesting an aerosol transmission mechanism and the need for disinfection protocols to include such surfaces.

Recent regulations proposed by the American Dental Association (ADA), the U.S. Centers for Disease Control and Prevention (CDC), and the U.S. Occupational Safety and Health Administration (OSHA) recommend that “all procedures involving blood or other potentially infectious materials should be performed in such a manner as to minimize splashing, spraying, spattering, andgeneration ofdroplets of these substances.” Thus, a device, system and method for capturing and containing aerosols such as bioaerosols and controlling the spatial distribution of the aerosols would be highly desirable. It is to such a device, system and method that the present disclosure is directed.

### DETAILED DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness.

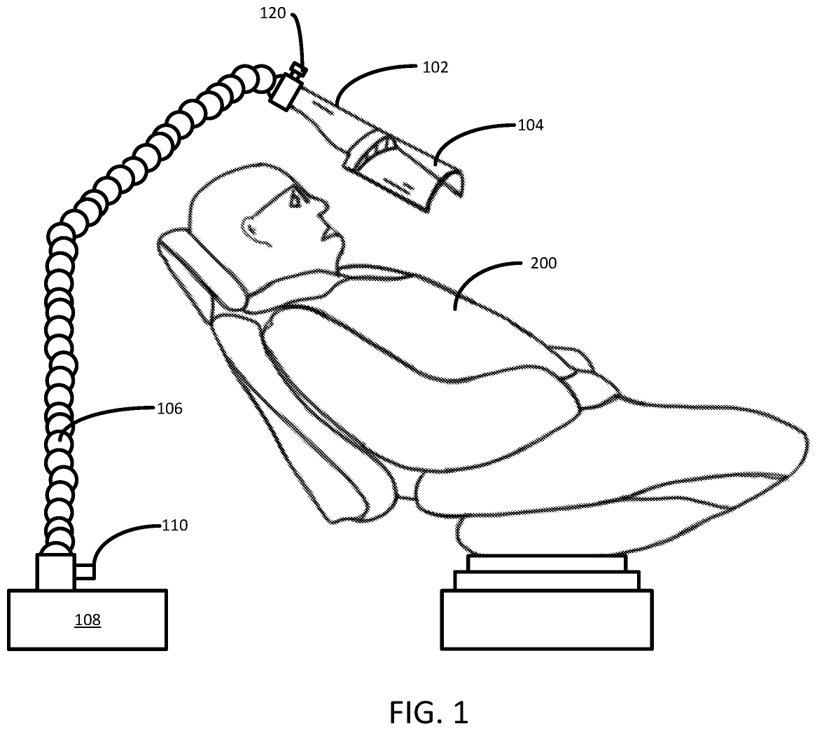

FIG. 1 depicts the use of an aerosol containment device with an adult dental patient.

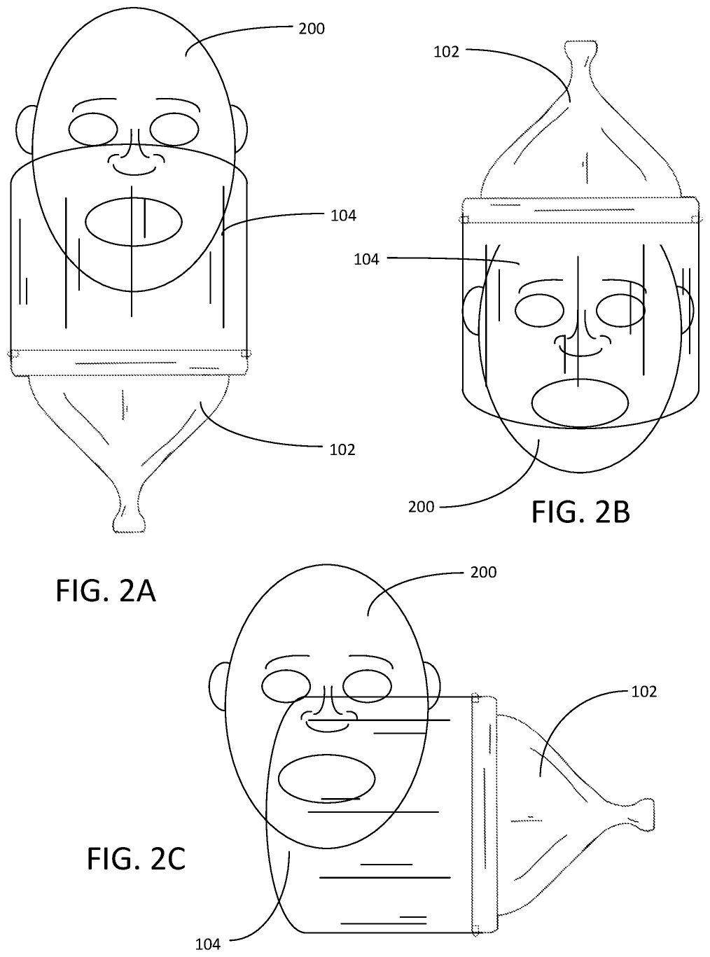

FIGS. 2A-2C depict several rotational orientations of an embodiment of the aerosol containment device over a dental patient.

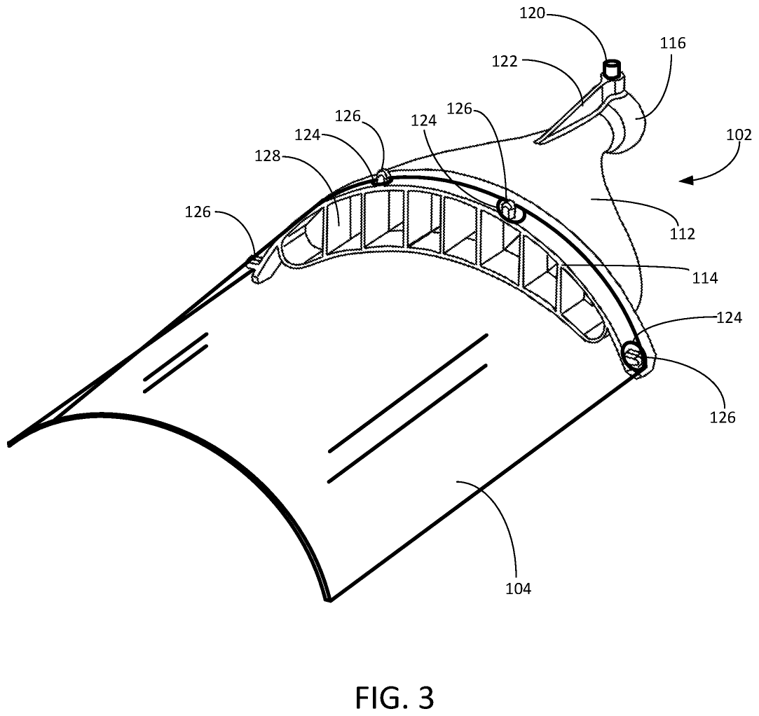

FIG. 3 depicts the manifold and shield of an embodiment of the aerosol containment device of FIG. 1.

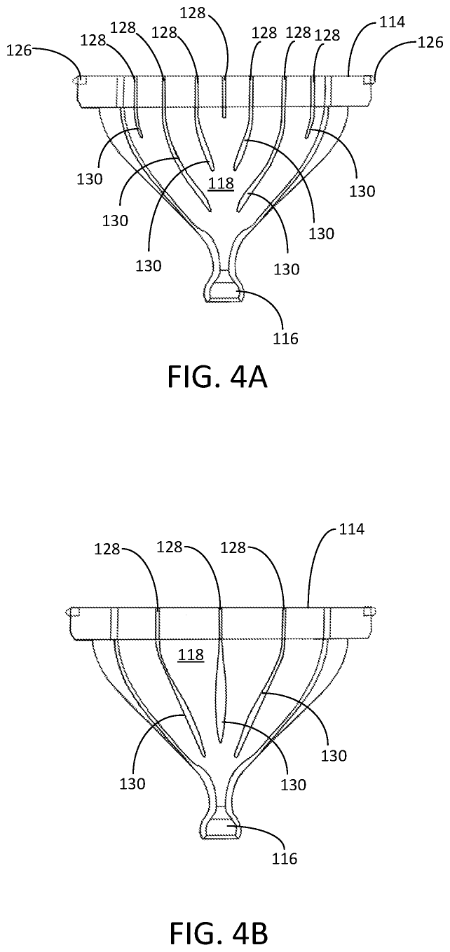

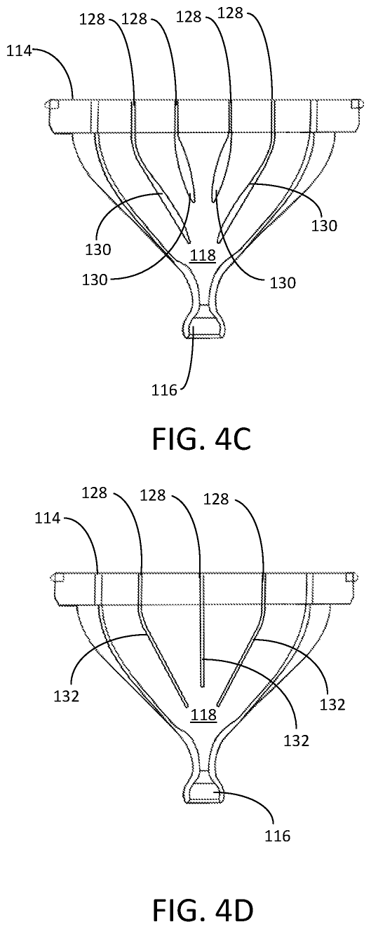

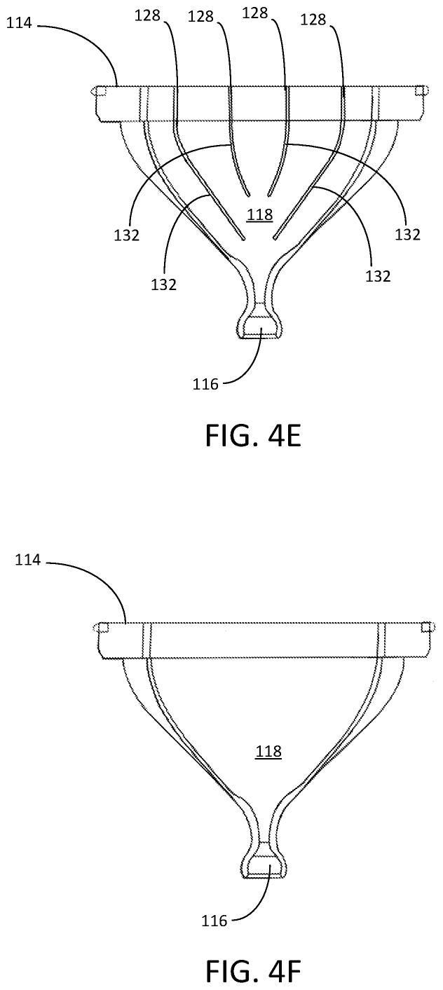

FIGS. 4A-4F depict several embodiments of the manifold of the aerosol containment device with different configurations of vanes and foils.

### DETAILED DESCRIPTION

The COVID-19 pandemic has imposed unprecedented occupational challenges for nurses, physicians and dentists. In dentistry, for example, handheld instruments such as air- and electric-handpieces, ultrasonic scalers and air/water syringes are capable of generating aerosols, droplets, and splatter, thereby exposing medical professionals to airborne contaminants such as viruses, bacteria and fungi. The present disclosure is directed to a novel aerosol containment device 100 for use in controlling the spatial distribution of aerosols containing inert fluorescent particles (e.g., particles having diameters in a range of about 0.30 μm to about 20.00 μm) such as in a dental clinic. In an experimental study, portable laser aerosol spectrometers were used to measure, in real-time, size-resolved number concentration of aerosols generated by a collision nebulizer. Results demonstrated that aerosol number concentrations were significantly decreased by using the aerosol containment device 100, which was able to efficiently decrease (e.g., by 8.56-fold) the number and size distribution of particles in a large clinic. The novel aerosol containment device 100 demonstrated higher efficiency for particles shown to contain the highest levels of SARS-CoV-2-in hospitals, thereby demonstrating the ability to decrease the spreading of nosocomial pathogens in medical settings, such as dental offices.

Before describing various embodiments of the present disclosure in more detail by way of exemplary description, examples, and results, it is to be understood that the embodiments of the present disclosure are not limited in application to the details of methods and apparatus as set forth in the following description. The embodiments of the present disclosure are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that certain embodiments of the present disclosure can be practiced without these specific details. In other instances, features which are well known to persons of ordinary skill in the art have not been described in detail to avoid unnecessary complication of the description.

Unless otherwise defined herein, scientific and technical terms used in connection with the embodiments of the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which embodiments of the present disclosure pertain. All patents, published patent applications, and non-patent publications referenced in any portion of this application are herein expressly incorporated by reference in their entirety to the same extent as if each individual patent or publication was specifically and individually indicated to be incorporated by reference.

While the methods and apparatus of the embodiments of the present disclosure have been described in terms of particular embodiments, it will be apparent to those of skill in the art that variations may be applied thereto and in the steps or in the sequence of steps of the methods described herein without departing from the spirit and scope of the inventive concepts. All such similar substitutes and modifications apparent to those skilled in the art are deemed to be within the spirit and scope of the systems as defined herein.

As utilized in accordance with the methods and apparatus of the embodiments of the present disclosure, the following terms, unless otherwise indicated, shall be understood to have the following meanings.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error. Further, in this detailed description, each numerical value (e.g., time or frequency) should be read once as modified by the term “about” (unless already expressly so modified), and then read again as not so modified unless otherwise indicated in context. The use of the term “about” or “approximately” may mean a range including ±0.5%, or ±1%, ±2%, or ±3%, or 44%, or ±5%, ±6%, or 77%, or ±8%, or ±9%, or 10%, or ±11%, or 12%, or 13%, or 14%, or +15%, or 25% of the subsequent number unless otherwise stated.

As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, or at least 90% of the time, or at least 95% of the time, or at least 98% of the time.

Features of any of the embodiments described herein may be combined with any of the other embodiments to create a new embodiment. As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50. Similarly, fractional amounts between any two consecutive integers are intended to be included herein, such as, but not limited to, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, and 0.95. For example, the range 3 to 4 includes, but is not limited to, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, and 3.95. Thus, even if specific data points within the range, or even no data points within the range, are explicitly identified or specifically referred to, it is to be understood that any data points within the range are to be considered to have been specified, and that the inventors possessed knowledge of the entire range and the points within the range.

Reference to a series of ranges includes ranges which combine the values of the boundaries of different ranges within the series. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range.

Thus, to further illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure.

The present disclosure will now be discussed in terms of several specific, non-limiting, examples, and embodiments. The examples described below, which include particular embodiments, will serve to illustrate the practice of the present disclosure, it being understood that the particulars shown are by way of example and for purposes of illustrative discussion of particular embodiments and are presented in the cause of providing what is believed to be a useful and readily understood description of procedures as well as of the principles and conceptual aspects of the present disclosure.

Turning to FIG. 1, shown therein is a depiction of the aerosol containment device 100 in use with an adult dental patient 200. The aerosol containment device 100 includes a manifold 102 and a shield 104 connected to the manifold 102. The manifold 102 is connected to a vacuum hose 106, which in turn is connected to a vacuum source 108. In exemplary embodiments, the vacuum hose 106 is a structured hose that is sufficiently rigid to support the weight of the manifold 102 and the shield 104, but flexible enough to permit manipulation to position the shield 104 and manifold 102 over the face of the patient 200.

The vacuum hose 106 can include an integrated valve 110 that can be used to separate the manifold 102 from the vacuum source 108. Suitable vacuum hoses 106 are commercially available under the Loc-Line trademark. The vacuum source 108 can be a conventional centralized vacuum system available in most dentist offices and operating theaters. In exemplary embodiments, the vacuum source 108 is a high volume evacuation system that is capable of creating an airflow through the manifold 102 of between about 50 and 300 liters per minute (1 μm). In some embodiments, the air flow through the manifold 102 is maintained at a rate of between 100 and 200 liters per minute. In some applications, the integrated valve 100 can be manipulated to achieve the most efficient flow rate through the manifold 102.

As better depicted in FIGS. 2A-2C, the aerosol containment device 100 is designed such that the shield 104 can be positioned above the face of the patient 200 in a number of ways to capture aerosols discharged from the nose or mouth of the patient 200. The shield 104 is configured to permit the dentist or other clinician to work under the shield 104, while maintaining visibility through the shield 104. Depending on the clinician's position relative to the patient 200, the aerosol containment device 100 can be shifted, lifted, rotated, and tilted to provide access to the patient 200 while capturing aerosols discharged from the patient 200. In some embodiments, the shield 104 is positioned between about 10 mm and about 50 mm above the face of the patient 200. The aerosol containment device 100 was designed to provide an inexpensive tool that is self-supported, infinitely adjustable, and effectively captures aerosols discharged from the patient 200 without requiring hands-on support by the dentist or clinician.

Turning to FIG. 3, shown therein is a perspective view of the manifold 102 and shield 104. The manifold 102 has a generally funnel-shaped body 112 that tapers from a front inlet 114 to a rear discharge 116. In some embodiments, the front inlet 114 has an elliptical or rectangular cross section, which can be arcuate or curved as depicted in FIG. 3. The body 112 is hollow and provides a plenum 118 for carrying air and aerosols from the front inlet 114 to the rear discharge 116. The rear discharge 116 is connected to the vacuum hose 106. In some embodiments, the position of manifold 102 relative to the vacuum hose 106 can be fixed by tightening a set screw 120 on the manifold 102. Releasing the force applied by the set screw 120 permits the clinician to adjust the position of the manifold 102 relative to the vacuum hose 106. A brace 122 can be used to support the set screw 120 and add rigidity to the body 112 of the manifold 102.

The manifold 102 can be manufactured from a lightweight metal, plastic, or composite material. In some embodiments, the manifold 102 is produced using additive manufacturing techniques with appropriate polymer materials. The shield 104 is composed of a transparent or translucent plastic. In some embodiments, the shield 104 is attached to the manifold 102 by aligning attachment apertures 124 on the shield 104 over shield tabs 126 on the manifold. This permits the shield 104 to be removed from the manifold 104 for cleaning or replacement. Other means for securing the shield 104 to the manifold 102 are also contemplated as falling within the scope of exemplary embodiments, and include hook-and-loop fasteners, magnets, adhesives, and tongue-and-groove arrangements.

Although the size of the manifold 102 and shield 104 can vary with application and patient 200, in a non-limiting example the manifold 102 has a length of about 387 mm, a width of about 188 mm and a height of about 70 mm. In this example, the manifold 102 is additively manufactured using polylactic acid (PLA). For this example of the manifold 102, the shield 104 has a width of about 238 mm and a length of about 200 mm. In alternate, but non-limiting embodiments, each of the dimensions of the device can be from 25% to 100% of those listed above (i.e., manifold length, width, or height, or shield width or length). In alternate non-limiting embodiments, the manifold 102 may have a length of 387.40 mm+100 mm, a width of 188.00 mm+100 mm, and a height of 69.90 mm 50 mm. The shield 104 may have a width of 238.12 mm+100 mm and a length of 200.00 mm 100 mm. In alternate non-limiting embodiments, the manifold 102 of the device may have a length in a range of 100 mm to 775 mm, a width in a range of 50 mm to 375 mm, and a height in a range of 15 mm to 150 mm. The shield 104 may have a width in a range of 50 mm to 475 mm and a length of 50 mm to 400 mm.

Turning to FIGS. 4A-4F, shown therein are plan views of the interior of the manifold 102. An important aspect of the aerosol containment device 100 is the internal configuration of the manifold 102, which is designed to optimize the passage of air and contaminants through the plenum 118 of the manifold 102. Beginning with FIG. 4A, the manifold 102 is provided with a series of entry vanes 128 and interior foils 130. The entry vanes 128 are substantially planar, while the interior foils 130 present a curved design that reduces turbulence of air passing through the plenum 118 of the manifold 102. As depicted in FIG. 4A, the manifold includes seven entry vanes 128 and four interior foils 130. In contrast, the embodiment depicted in FIG. 4B includes three entry vanes 128 that are each connected to a corresponding interior foil 130, and the embodiment depicted in FIG. 4C includes four entry vanes 128 that lead to four interior foils 130.

Unlike the embodiments depicted in FIGS. 4A-4C, the embodiment depicted in FIG. 4D has three entry vanes 128 that lead to three interior vanes 132. Unlike the interior foils 130 depicted in the embodiments of FIGS. 4A-4C, the embodiment depicted in FIG. 4D does not include interior foils 130. Similarly, the embodiment depicted in FIG. 4E also includes four entry vanes 128 and four interior vanes 132. The embodiment depicted in FIG. 4E does not include an interior foil 130. As used herein, the term “flow features” refers generally to one or more of the entry vanes 128, the interior foils 130 and the interior vanes 132.

The entry vanes 128, interior foils 130 and interior vanes 132 are positioned vertically within the plenum 118. The entry vanes 128 and interior vanes 132 each have a substantially constant thickness or cross-section, while the interior foils 130 have a variable thickness or cross-section. For example, the thickness of the rear end portion of the interior foils 130 may be greater than the thickness of the front of the interior foils 130. In certain non-limiting embodiments, the number of entry vanes 128, interior foils 130, and interior vanes 132 may vary from 1 to 20, for example 1, 2, 3, 4, 5, 6, 7, 8, 9, 10, 11, 12, 13, 14, 15, 16, 17, 18, 19, or 20, or more.

In some embodiments, the manifold 102 does not include any interior foils 130 or interior vanes 132. The embodiment depicted in FIG. 4F does not include any entry vanes 128, interior foils 130, or interior vanes 132. This provides an unobstructed plenum 118 that may perform well in applications where the flow rate through the manifold 102 is low and less likely to create disruptive turbulence.

Thus, the aerosol containment device 100 of the present disclosure includes a flattened funnel-shaped body 112 having a wide front inlet 114 and a narrow rear discharge 116 on the opposite side of the body 112. The interior of the body 112 defines a plenum 118, which is tapered from the front inlet 114 to the rear discharge 116, which is configured for an adjustable connection to the vacuum hose 106. The body 112 may contain a plurality of entry vanes 128, interior vanes 132 and interior foils 130, which extend from the front inlet 114 towards the rear discharge 116.

The aerosol containment device 100 has been thoroughly tested in clinical scenarios. These studies have demonstrated, for the first time in dentistry, that particles similar in size to those found in hospitals to have highest concentration of SARS-CoV-2 (e.g., 0.30 μm-0.40 μm) are capable of spreading throughout the entire area of a large operatory room in a dental school. The aerosol containment device 100 was found to decrease detected particle concentrations by up to 8.56-fold. The efficiency of the aerosol containment device 100 was shown to be higher for particles with dimensions that are SARS-CoV-2-like, and therefore, the aerosol containment device 100 can be expected to decrease the spread of infectious respiratory diseases in medical settings such as dental settings, or any other settings where infective agent-bearing aerosols may be created.

More specifically, the testing indicates that the aerosol containment device 100 drastically reduced the concentration of particles having dimensions similar to those containing SARS-CoV-2 (0.30 μm-1.00 μm) in hospitals. It was also observed that concentrations detected tended toward zero with or without the utilization of the aerosol containment device 100 above a specific particle diameter threshold (about 3 μm). Taken together, these results suggest that particles smaller than about 3 μm are generated far more abundantly than larger particles and that smaller particles (<3 μm) are transported more efficiently than larger particles (>3 μm). Based on results reported it was possible to rank order particle distribution behavior as follows: 0.30 μm-0.50 μm >0.50 μm-1.00 μm >1.00 μm-3.00 μm >4.00 μm-5.00 μm >5.00 μm-20.00 μm In general, the results of the size-resolved spatial distribution analysis disclosed herein demonstrate that the use of the aerosol containment device 100 reduced the airborne spread of small particles of greatest concern for COVID-19 transmission in dental settings. The distance-dependent spreading behavior of particles was observed to vary inversely with particle diameter (0.30 μm >0.40 μm >0.50 μm >0.65 μm >0.8 μm >1.00 μm >1.60 μm >2.00 μm >3.00 μm >4.00 μm >5.00 μm >7.50 μm >10.00 μm >15.00 μm >20.00 μm).

Thus, the embodiments of the present disclosure are well adapted to carry out the objects and attain the ends and advantages mentioned above, as well as those inherent therein. While the inventive aerosol containment device 100 has been described and illustrated herein by reference to particular non-limiting embodiments in relation to the drawings attached thereto, various changes and further modifications, apart from those shown or suggested herein, may be made therein by those of ordinary skill in the art, without departing from the spirit of the inventive concepts.

It is claimed:

1. An aerosol containment device for use in containing aerosols discharged during a dental procedure, the aerosol containment device comprising:
a manifold; and
a shield connected to the manifold.

2. The aerosol containment device of claim 1, wherein the manifold comprises a body that has:
a front inlet;
a rear discharge; and
plenum extending between the front inlet and the rear discharge.

3. The aerosol containment device of claim 2, wherein the manifold comprises one or more entry vanes adjacent to the front inlet, wherein each of the one or more entry vanes has a cross-section that is substantially constant.

4. The aerosol containment device of claim 3, wherein the manifold comprises between two and eight entry vanes.

5. The aerosol containment device of claim 3, wherein the manifold comprises one or more interior foils inside the plenum, wherein each of the one or more interior foils has a cross-section that is variable.

6. The aerosol containment device of claim 5, wherein the manifold comprises between two and eight interior foils.

7. The aerosol containment device of claim 3, wherein the manifold comprises one or more interior vanes inside the plenum, wherein each of the one or more interior vanes has a cross-section that is substantially constant.

8. The aerosol containment device of claim 7, wherein the manifold comprises between two and eight interior vanes.

9. The aerosol containment device of claim 2, wherein the manifold further comprises a plurality of attachment tabs and wherein the shield comprises a plurality of attachment apertures that are configured to capture a corresponding one of the plurality of attachment tabs to secure the shield to the manifold.

10. The aerosol containment device of claim 2, wherein the shield is manufactured from a clear plastic.

11. The aerosol containment device of claim 2, further comprising:
a vacuum source; and
a vacuum hose connected between the vacuum source and the rear discharge of the manifold.

12. The aerosol containment device of claim 11, wherein the manifold further comprises a set screw that is configured to selectively hold the manifold in fixed positional relationship with the vacuum hose.

13. The aerosol containment device of claim 12, wherein the vacuum hose is a structured hose.

14. An aerosol containment device for use in containing aerosols discharged during a dental procedure, the aerosol containment device comprising:
a vacuum source;
a vacuum hose connected to the vacuum source;
a manifold connected to the vacuum hose; and
a shield connected to the manifold, wherein the shield is manufactured from a transparent plastic.

15. The aerosol containment device of claim 14, wherein the manifold comprises a body that has:
a front inlet;
a rear discharge; and
plenum extending between the front inlet and the rear discharge.

16. The aerosol containment device of claim 15, wherein the manifold comprises a plurality of entry vanes adjacent to the front inlet.

17. The aerosol containment device of claim 16, wherein the manifold comprises a plurality of interior foils inside the plenum, and wherein each of the plurality of interior foils is attached to a corresponding one of the plurality of entry vanes.

18. The aerosol containment device of claim 16, wherein the manifold comprises a plurality of interior vanes inside the plenum, and wherein each of the plurality of interior vanes is attached to a corresponding one of the plurality of entry vanes.

19. The aerosol containment device of claim 14, wherein the manifold further comprises a set screw that is configured to selectively hold the manifold in fixed positional relationship with the vacuum hose.

20. An aerosol containment device for use in containing aerosols discharged during a dental procedure, the aerosol containment device comprising:
a vacuum source;
a vacuum hose connected to the vacuum source, wherein the vacuum hose is a structured hose;
a manifold connected to the vacuum hose, wherein the manifold comprises a body that has:
a front inlet;
a rear discharge connected to the vacuum hose;
a plenum extending between the front inlet and the rear discharge; and
a plurality of flow features within the plenum; and
a shield connected to the manifold, wherein the shield is manufactured from a transparent plastic.
